(19) United States
(12) Patent Application Publication
(10) Pub. No.: US 2007/0140426 A1
(43) Pub. Date: Jun. 21, 2007

(54) Treatment of lesions or imperfections in mammalian skin or near-skin tissues or in or near other anatomic surfaces

(76) Inventors: Steve Axelrod, Los Altos, CA (US); Paul A. Lovoi, Saratoga, CA (US)

Correspondence Address: THOMAS M. FREIBURGER, P.O. BOX 1026, TIBURON, CA, 94920, US

(21) Appl. No.: 11/607,811

(22) Filed: Dec. 1, 2006

Related U.S. Application Data
(60) Provisional application No. 60/742,118, filed on Dec. 2, 2005.

(51) Int. Cl. A61N 5/10

(52) U.S. Cl. 378/65

(57) ABSTRACT

A method of treatment for lesions or imperfections in or near exposed anatomic surfaces such as skin using low-level ionizing radiation includes, in one embodiment, acquisition by computer of the location and geometry of the anatomic region to be treated, creation of a treatment plan by a therapist to achieve the desired therapeutic effect within the region, and execution of the treatment plan by the energy source. Verification of the treatment to plan and safety methods are disclosed.

This application claims benefit of provisional application No. 60/472,118, filed Dec. 2, 2005.

### BACKGROUND OF THE INVENTION

This invention relates to the field of mammalian therapy by means of ionizing radiation or laser energy applied to tumors or other imperfections in skin or near-skin tissues, or in or near other exposed anatomical surfaces.

A variety of skin or near skin medical conditions are today treated by application of x-ray, laser, or electron radiation, often after surgery to excise a tumor or other defect. Generally, the radiation treatment apparatus used for these purposes is large, expensive and unwieldy. As such, it is often unavailable in small clinic situations. Furthermore, the radiation dosage level is often imprecise in that the energy ranges available to the therapist are too great for the desired treatment, necessitating that the tumor be covered with radiation absorbing material such that the radiation passing through the absorber is more appropriate for the desired treatment. Because of the high-energy radiation levels from the machine, the patient must be extensively shielded except for the area of the tumor.

Further imprecision of current methods derives from the fact that available radiation machines have fixed output patterns which must be extensively collimated by magnetic fields and secondary shielding, often prepared specifically to treat the individual patient's tumor. Although machine output may be adjusted in a gross sense, the radiation distribution within the area of the collimated beam which is allowed to pass through the mask to treat the tumor is uncontrolled. The therapist must therefore compromise his or her treatment plan to accommodate an average energy level when it would be preferable from a therapeutic viewpoint to tailor the treatment energy levels for specific sub-regions of the tumor area.

In use, the machine is positioned over the patient; more specifically, the collimated beam passing through the secondary shielding is directed at the subject tumor site. The patient must not move once alignment is determined, otherwise the treatment is not delivered to the desired area. When all are ready, the therapist leaves the room, and treatment progresses. This situation can be intimidating or claustrophobic for the patient.

Present methods of laser therapy on surfaces is often by direct application CO₂ laser energy, or by injection of agents which are attracted to and linger in abnormal tissue cells. When activated by laser, these agents are activated, destroying the abnormal tissue (e.g. Photofrin®, Axcan Pharma United States, Birmingham, Ala.). With present methods of laser therapy, the therapist must often rely on visual feedback to determine the region of treatment, and the extent or intensity of treatment within that region. Although the therapist may know quantitatively the energy required to achieve the desired therapeutic effect, the visual clues available may not accurately reflect the extent of treatment in a useful way. Hence there may remain uncertainty as to the adequacy of treatment. This in turn can lead to untreated areas within the region of interest, over-treatment, or return treatments after the effects of initial treatment become apparent.

There is therefore need for apparatus and methods for accurately and conveniently treating tumors and imperfections in surface or near-surfade tissues. It is an object of this invention to provide methods for identifying the region to be treated on the patient, planning the therapy, or radiation dose, quantitatively to be administered within that region, taking into account the known characteristics of the energy source to be utilized, and executing the therapy conveniently, and in a timely manner. Additionally, it is an object of this invention to provide a record of the therapy delivered, and verifying it was to plan. Specifically, this invention is directed to the use of small ionizing radiation sources, facilitating such radiation therapy in small clinical settings.

### SUMMARY OF THE INVENTION

The preferred method of this invention includes mapping the region of therapeutic interest by a computer equipped with imaging apparatus and displaying the region on a monitor. Imaging apparatus of this sort are well known; for example, digital video camera systems are useful. Preferably, the imaging apparatus is stereoscopic or includes known methods of triangulation, for example by laser (LMI Technologies, Inc., Delta, British Columbia, Canada), such that the range from the energy source to the surface areas within the treatment region can be deduced. This is especially important for determining the dose delivered where the treatment region is not substantially flat, or is extensive. To accomplish this mapping, the therapist may outline the region physically on the patient, for example by a marker pen, such that the region may be automatically scanned, or by direct acquisition by the computer based on imaging the tumor or other defect by its characteristics, and outlining of the region of interest by the therapist on the monitor, for example by known mouse or tablet and pen-based methods (Wacom Technologies Corp., Vancouver, Wash.).

With this image and the treatment region boundaries defined, the therapist can plan his course of treatment, for example again by tablet and pen-based input for the local dose needed. Computer smoothing of discrete input data over the region can be applied as desired. Dose delivered may be deduced from energy source input parameters, for example voltage and current in the case of an x-ray source, and proximity sensors (for example laser triangulation) to determine range from energy source to the treatment surface, to calculate the energy flux incident on the surface being treated, and cumulative dwell over each location. When the desired treatment level has been reached for a given location, or when the beam passes outside the treatment region boundary, the beam of energy incident on that area may be interrupted.

Verification can be in real time, with radiation sensors adjacent to the target tissue. These may be in the periphery of the radiation beam, or directly on the tissue region, and their feedback can be used to control the delivery of energy to the region or to subregions of the region.

The energy source may be automatically or manually scanned, aiming the source at the treatment area in sequential steps or continuously, methodically or randomly, in order to deliver the planned therapy. Continuous imaging of the area around the incident radiation and comparison with the original mapping accurately establishes the location of incident radiation within the treatment area. Computation of the cumulative dose by location (or real time incident radiation measurement) is used both to display the progress of treatment and to control radiation delivered. Apparatus capable of real-time image recognition of this sort is well known, and not unlike that for missile location determination, although obviously at much shorter range (National Instruments, Austin, Tex.). Recording the delivered dose locally provides verification that the planned dose was achieved. As an adjunct or alternative to computer generated verification of dose delivered, and as noted above, in-beam dosimeters can be used but can present the disadvantage of creating a shadow in the incident radiation unless placed in peripheral areas of the beam. Surface mounted dosimeters could be used in place of, or in conjunction with the methods described, and local shadowing can be overcome by embedding the dosimeters in openings in an attenuating shield having the same shielding density as the dosimeters.

Although this invention is largely described with respect to therapeutic application of ionizing radiation, the same planning and treatment control, turning the energy source on and off as planned therapy is achieved, is equally applicable to laser therapy. It is particularly useful where any surface effects from treatment visually obscure the extent of treatment and therefore limit the therapist's ability to optimally conclude treatment.

### DESCRIPTION OF THE DRAWINGS

FIG. 1 shows schematically the system of the invention in relation to a portion of patient anatomy.

FIG. 2 shows the energy beam cone of therapeutic intensity and the cone of image recognition.

FIG. 3 shows a cross section of an x-ray energy source which may be used in this inventions with a coaxial camera mounted thereon.

FIG. 4a shows a shutter mechanism capable of interrupting the energy beam in the closed position.

FIG. 4b shows the shutter mechanism in the open position.

FIG. 5 is a side view, which may be a side elevation view, of a radiation treatment system treating a region of a patient, such as a portion of the skin surface.

FIG. 6 is a similar view, but showing another embodiment.

FIG. 7 is a plan view of a flexible absorber forming a part of the system of FIG. 6.

### DESCRIPTION OF PREFERRED EMBODIMENTS

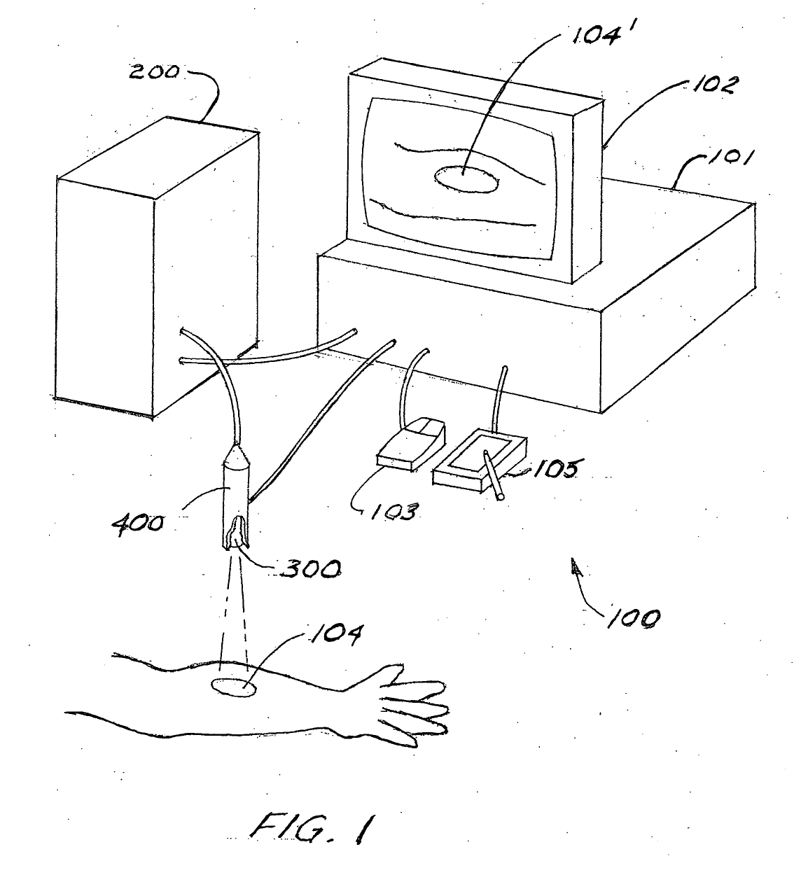

FIG. 1 shows the apparatus of the invention for radiation treatment with low-energy x-ray in schematic view. A central controller 100, with a CPU 101, a mouse 103, a monitor 102, and a tablet and pen 105 input device, is in communication with a power source 200, supplying high voltage power and in one embodiment a source of laser energy, for purposes of controlling the x-ray energy source 300 with regard to penetration depth and intensity. In another embodiment, laser energy is used to provide markers to assist in image recognition. The controller 100 is also in communication with an imaging camera 400 in order to receive coherent images of the treatment site. The camera 400 is located near or about radiation energy source 300 such that it images the area being treated.

The preferred method of use of this apparatus comprises first imaging the patient at least once using a relatively wide exposure angle at a known distance from the treatment surface such that the treatment region, and somewhat beyond, is included in good detail and displayed on the monitor 102. Detail is necessary for both location recognition and ranging as described below. If the region 104 to be treated is sufficiently non-planar, it may be desirable to take more than one wide angle image. Next the treatment region 104 is identified for acquisition by the CPU 101. If the treatment region was defined on the patient physically, this region is displayed on the monitor 102 as 104. If not identified physically on the patient, the treatment region 104 may be indicated on the monitor 102 display using, for example, tablet and pen-based input means. With the treatment region defined, the operator may then proceed to assign treatment parameters (dose) within that region, again by tablet and pen-based means. Other types of input methods might also be used.

In a preferred method of this invention, the radiation energy source utilized is disclosed in U.S. Pat. No. 6,319,188, “Vascular X-Ray Probe”, adapted as to power and for forward or distal projecting radiation. The specification of U.S. Pat. No. 6,319,188 is incorporated herein by reference in its entirety. The energy source 300 may be a hand held instrument, comprised for example of a central energy source 300 and an adjacent or coaxial camera 400 near or surrounding the source. The energy source 300 is in communication with the power source 200 to receive the voltage, current and, in one embodiment, laser energy necessary to deliver controlled radiation energy to each sub-region within the treatment region 104 in accordance with the treatment plan. The adjacent or coaxial camera 400 is in communication with the CPU 101 in order to provide the wide angle image of the patient used in preparation of the treatment plan noted above, and to continuously match shorter range images with the wide angle view used to create the treatment plan within the treatment region 104. By identifying and matching landmarks (natural or artificial) on the wide angle and treatment images, the location of the incident radiation can be determined. Further, by comparing the scale between imaged landmarks proportionally during treatment to the scale between the same landmarks on the wide angle image most normal to the treatment surface, range from the energy source 300 to the treatment surface may be deduced and serve as a real-time basis for voltage and current adjustments to the energy source, modulating radiation output to provide incident radiation as planned. In this manner, the cumulative incident radiation and dwell can be matched to the total treatment planned for that location. Alternatively, other proximity determination methods could be used, such as laser triangulation. In an intra-operative, sterile context, independently guided laser spots could be used to provide reference landmarks.

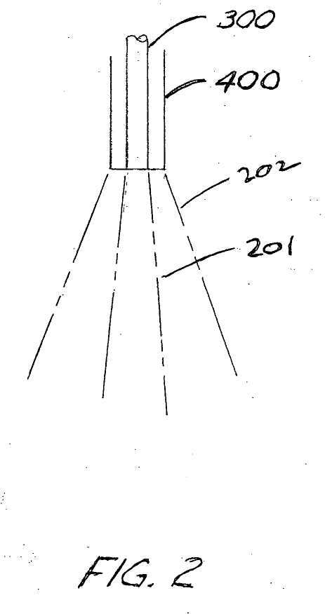

FIG. 2 shows the energy source 300 and its cone of therapeutic radiation 201 as well as the coaxial camera 400 and its imaging cone 202. The radiation cone is narrower than the visualization cone 202. The radiation cone is more focused in order to control delivery of radiation to sub-regions within the overall treatment region. The imaging cone is greater in order to encompass landmarks outside the radiation cone for determination of energy beam location. Comparison of angles read by the camera images between landmarks in wide angle and treatment range images can be used to deduce range between the energy source and treatment surface. The comparisons can also be used to indicate when the angle between the incident treatment beam 201 and the surface being treated is outside a desired range by noting distortion of the region boundary, or by changes in angular relationships between landmarks. This angular range will depend on the treatment plan and energy source parameters selected. A desirable angular range from normal is from 0° to 15°. The comparison feedback between wide angle and treatment images is used to modulate energy output or to indicate need for range or angular correction between the energy source 300 and the treatment surface, including particularly within region 104.

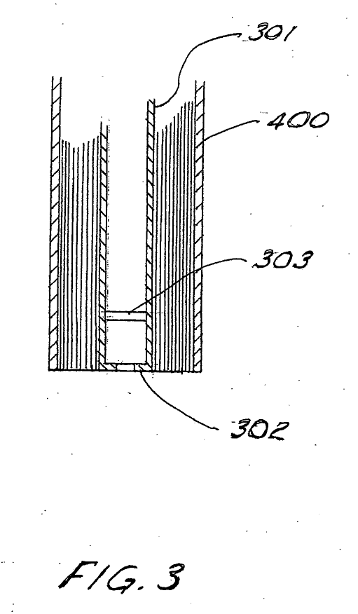

FIG. 3 shows a cross-section of the tip of the radiation energy source 300 and coaxial camera 400. The energy source 300 comprises a cylindrical energy source 301 vacuum envelope, a distal collimator 302 and a flat, transmission type anode 303. This arrangement can be proportioned to produce the narrow beam shown in FIG. 2. The coaxial camera 400 surrounds and is secured to energy source 300. It comprises a coherent, flexible bundle of glass fibers 401 as is common in medical imaging, but tubular in this case to accommodate the central energy source. Outside of this bundle is a flexible sheath 402 to provide environmental protection and flexibility, and can be, for example, a sheath of polyurethane. Alternatively, the camera 400 may not be tubular and may be mounted adjacent the energy source 300. The energy source 300 and camera 400 assembly may be hand held for random or manual scanning, or it may be mounted on an x-y (or x-y-z) stage for automated scanning over the treatment region 104.

If desired, calibration or verification of system radiation output before and after treatment can be provided using known flat-panel x-ray detector, ion chamber array, or film methods. This would provide real verification that the energy source 300 is in good order before starting treatment, and verification that treatment was to plan.

When the therapist is ready to administer therapy to the patient, the energy source 300 is enabled at the controller 100 such that radiation can be delivered when positioned over the treatment area 104 in accordance with the treatment plan, and when the energy source 300 is activated. Activation can advantageously be by push button on the energy source 300 and camera 400 assembly, or alternately by foot pedal, for example.

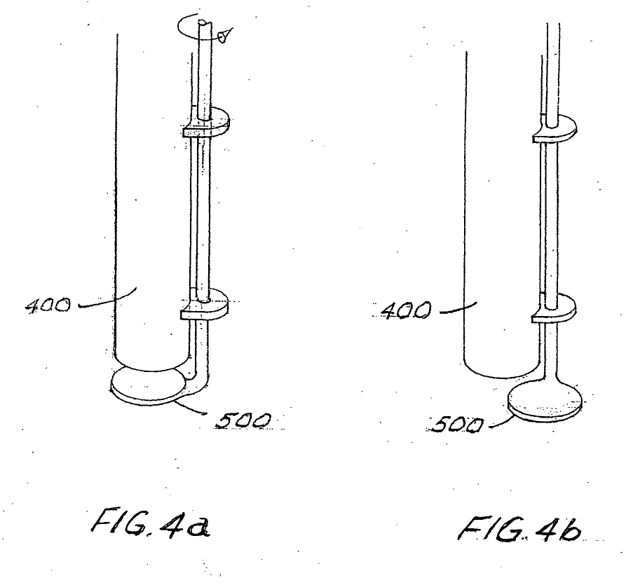

As the energy source 300 is scanned during the treatment phase, whether by manual or automated protocol, the controller 100 computes the incident level of treatment by specific sub-region using range and energy source output to integrate the cumulative treatment over time until the cumulative dose reaches the treatment plan level for that sub-region, at which point the energy beam is blocked. This may be accomplished by an operable shutter 500 as depicted in FIGS. 4a and 4b, made for example from steel or tungsten, which may be used to block the energy beam from reaching the region of treatment. As an alternative to a shutter, the beam may be switched off by cutting off the high voltage to the energy source 300, reducing the high voltage to a level below, for example, 10 kv, cutting cathode (filament) power, or reducing cathode temperature to below the emission threshold. Similarly, if the energy beam is scanned beyond the treatment region 104, or if the incident beam angle onto the treatment surface passes outside the acceptable range, the energy beam is also blocked or switched off until it is again properly directed within the treatment region to a position still requiring treatment.

As noted above, when the prescribed level of therapy has been achieved, or when the beam leaves the defined treatment region, the shutter 403 will close or the beam will switch off, and a signal can be used to advise the therapist to move to an area still in need of further treatment. Signaling might be, for example, by coloring the area on the monitor within the treatment region which has received adequate treatment, perhaps in an analog manner such that the darker the color, the more nearly complete the treatment in that area. Optionally, after complete treatment of a sub-region, the color could shift to a different color altogether. The operator can also be notified the beam is off by an audible signal. Should the therapist later again direct the beam to an area already adequately treated, or leave the treatment region, the shutter will again close (or radiation will be switched off) in order to prevent over-treatment. Where manual scanning is used, audible signals can be useful in guiding the therapist within a proper distance range from the treatment surface. For example, a low pitched tone or fast beeping signal could indicate the energy source 300 is too close to the surface, whereas a high tone or slow beeping would indicate too great a range. Typical treatment ranges might be from 0.1 to 10 cm between the energy source 300 and treatment surface. Tones can also be used to indicate percentage completion of planned treatment by specific region. A record of a fully dark colored, or different colored treatment region on the monitor can serve as verification that the planned treatment has been delivered. In the case of automated scanning, signaling may not be necessary. However, a record of the darkened or differently colored image on the monitor can still be useful as verification of treatment to plan. Alternatively, as treatment progresses, the fully treated areas can be eliminated from the treatment region on the monitor, leaving only those areas still in need of further treatment.

A further hand-held embodiment includes a stage over the treatment region on which a plate, with the energy source mounted normal to the plate, slides when moved by hand. Such apparatus can both decrease operator fatigue and assure the range between the energy source and treatment region and the incident angle are known.

Although this method is described in relation to low-energy x-ray sources, it may in principle be applied to other energy sources, non-x-ray, or to higher power sources. This method eliminates potential treatment error due to patient movement because, by actively and continuously acquiring camera images of the treatment region, it delivers therapy to the treatment region where the patient is in real time, rather than to where the patient was during creation of the treatment plan, or at some subsequent point. With this method, therapy to a predetermined plan can be accomplished and verified, and ensuring that over-treatment is eliminated, thus assuring a safe therapeutic effect.

The system and method of this invention is less elaborate and more space efficient than an automated system, making it more attractive in a small clinic setting. It is less costly, is easier to use for the therapist and less intimidating to the patient.

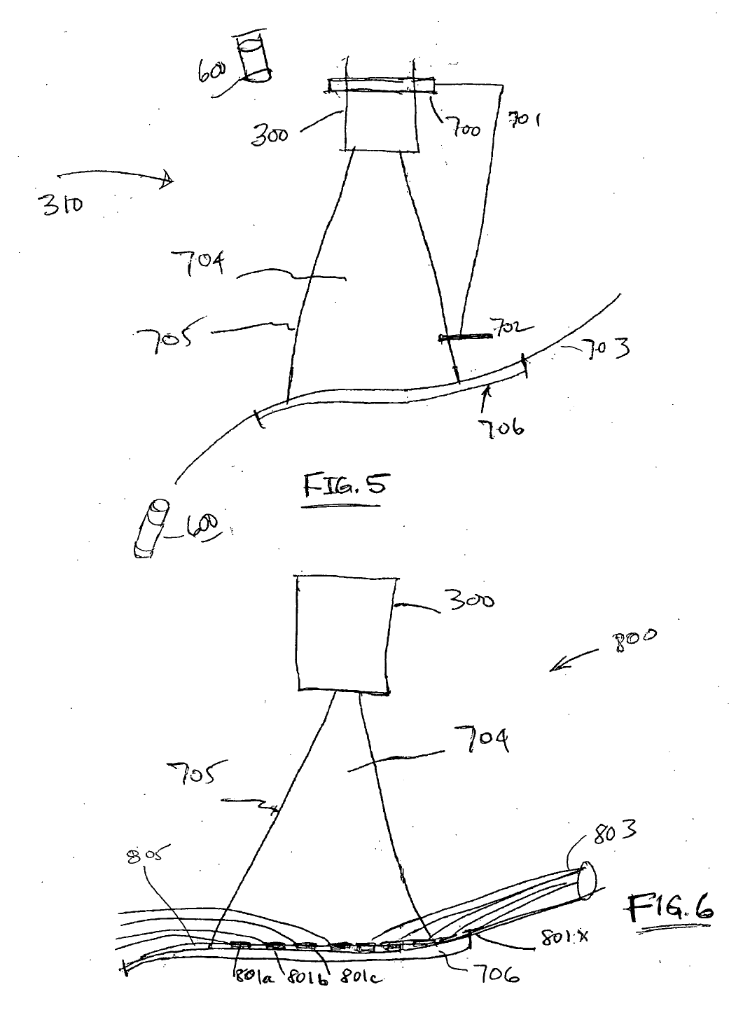

FIG. 5 is a side view of another embodiment of a radiation treatment system 310 which is capable of more direct real-time monitoring and verification. The drawing shows an x-ray source or other radiation source 300 that is collimated (or delivered as a slightly diverging beam) into a treatment beam 704 generally bounded by rays 705. The treatment beam 704 is treating a treatment surface 703 containing a treatment region 706 (partially shown as a surface, as in a convexly curved anatomical surface), which may be on the skin or just under the skin of a patient. The treatment source 300 is moved over the treatment region 706 to provide radiation treatment over the entire region. A radiation detector 702 is held at least partially within the treatment beam 704 by a mechanical connection 701 that is in turn attached to the radiation source 300 or its connected structure and designed to move in cooperation with the entire treatment system 310. The detector 702 samples the radiation delivered by the treatment beam 705 to determine the total delivered radiation to the treatment region 706. The treatment plan prepared by the radiation physicist or the radiation oncologist determines the dose to be delivered to the region or sub-region.

The delivery system needs to deliver the treatment to the region without under- or over-treating any part of the region and minimizing the dose delivered outside the region (801x). Radiation is delivered to the treatment region and is monitored by the radiation detector 702. The location of the radiation treatment system (i.e. the source 300) preferably is monitored by a camera 600. Combining knowledge of the radiation delivered by the radiation source using detector 702, the location of the treatment system and how long the radiation system dwells at each location within the treatment region allows the controller to display the amount of treatment delivered to each of the areas within the region. The information can then be displayed on the monitor to show where the region has been under-, over- and correctly treated. Using a radiation detector that is at the fringe of the radiation minimizes the detector shadowing of tissue to be treated. This approach depends on measuring the radiation beam distribution and knowing the distance of the detector from the treatment surface.

It should be understood that other means of determination of the location of the radiation treatment system 310 can be used. For example, the location can be known through mechanical indexing of the position of the radiation source, in the event such an automatic indexing system is used, rather than hand-controlled movement.

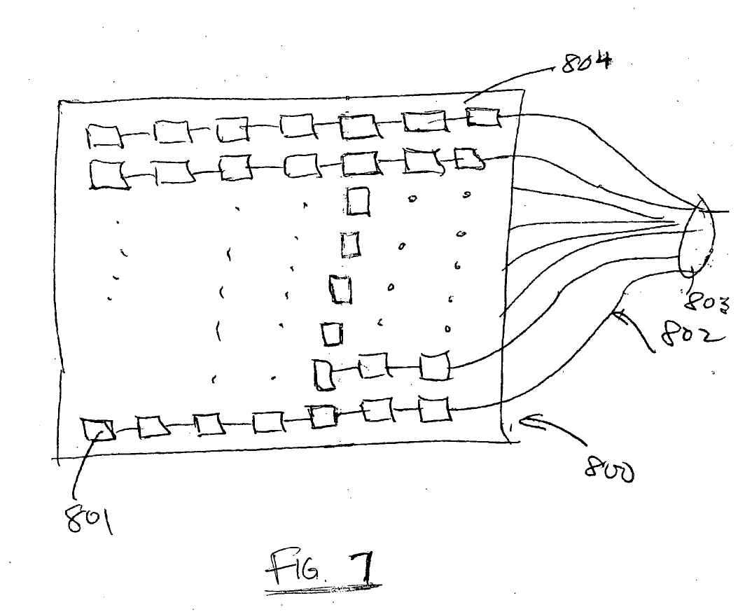

FIG. 6 shows another embodiment 800 of the invention where radiation detectors 801a, 801b, 801c, etc. are held in an array in a flexible sheet of material 805 that absorbs essentially the same amount of radiation as the detector. The detectors are embedded in openings in the sheet. In this way the attenuation of radiation due to absorption is made nearly or essentially uniform over the entire region.

FIG. 7 shows a plan view of the array of radiation detectors 801a, 801b, etc. in a flexible absorber. Each of the detectors, 801a, 801b, 801c, is connected by wires 802 to form a cable 803 for connecting to the controller (not shown). The controller integrates the dose received by each detector interpolates between detectors as needed, and displays the dose received on the display and calculates the dose remaining to be delivered. The dosimeters can be wirelessly connected to the controller if desired.

All of the above procedures and equipment can be used in connection with image re-acquisition software, as mentioned above, whereby a camera, in a larger field than the treatment area, constantly monitors either the treatment area or the larger portion of the patient around the treatment area and, in the event of movement of the patient, re-acquires the image and corrects the position of the radiation source accordingly.

It should be understand that the radiation, although often described above in terms of an electronic x-ray source, can alternatively comprise other ionizing radiation or even laser radiation. Other ionizing radiation can comprise, for example, electronic beam radiation, alpha particles, beta particles or protons in the case of x-ray radiation, preferably a miniature, controllable electronic x-ray source is used, at a voltage in the range of about 10 kV to 70 kV, more preferably about 10 kV to 30 kV.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A method for treating conditions of the skin or near-skin tissue with radiation, comprising:
defining a region to be treated,
imaging the region to be treated,
displaying the image of the region to be treated on a monitor connected to a processing unit,
preparing a treatment plan for irradiation of the region based on a prescription dose of radiation,
in the processing unit, inputting a treatment plan for irradiation of the region using a low-voltage electronic source of ionized radiation,
imposing on the image of the region on the monitor an overlay with indication of the treatment plan for the region,
initiating radiation treatment of the region using the electronic source, under the control of the controller and in accordance with the treatment plan,
monitoring and verifying treatment by computing the incident level of radiation treatment on the region using range to tissue of the region and the electronic radiation source output level and integrating over time until cumulative dose received throughout the region reaches the treatment plan level, and
discontinuing irradiation when the treatment plan level of incident radiation is reached as determined by the computing step.

2. The method of claim 1, wherein the region to be treated is divided into sub-regions, and shown divided into sub-regions on the monitor.

3. The method of claim 2, wherein the irradiation treatment is conducted sub-region by sub-region, with sub-region being treated until the dose received throughout the sub-region reaches the treatment plan level, at which point irradiation is discontinued at that sub-region, and the method includes treating a series of sub-regions in succession.

4. The method of claim 1, further including, during treatment, continuously re-acquiring the image of the region to be treated including during any movement of the region due to movement of the patient, using an image acquisition program run by the controller.

5. The method of claim 1, wherein the step of defining a region to be treated includes marking the skin with a marker to visually indicate the region.

6. The method of claim 1 wherein a pen and tablet input device is connected to the controller, and the method including using the pen and tablet device to outline the treatment region on the monitor.

7. The method of claim 1, wherein a light pen, mouse, touch screen or other pointing device is connected to the controller, and the method including using the pointing device to outline the treatment region on the monitor.

8. The method of claim 1, wherein the source of ionized radiation is an electronic x-ray source with voltage in the range of about 10 kV to 70 kV.

9. The method of claim 1, further including, using the controller, imposing on the image of the region one or more color indicators in areas of the region, with color change to indicate to the physician or observer progress of the treatment by dose of radiation received in the region.

10. The method of claim 1, further including determining the dose delivered to the region being treated by means of a real-time radiation detector.

11. The method of claim 10, wherein the radiation detector comprises an array of detectors positioned directly over and in close proximity to the region to be treated.

12. The method of claim 11, wherein the radiation detectors are embedded in a sheet of plastic that has approximately the same radiation absorption as the detectors.

13. The method of claim 10, wherein the radiation detector is just outside the region being treated.

14. The method of claim 10, wherein the radiation detector is kept just outside the treatment perimeter of the radiation device.

15. A method for treating a surface or near-surface skin conditions, comprising:
defining the region to be treated on a patient,
translating the treatment region to a dose planning system on a computer,
initiating radiation treatment of the defined region,
monitoring the progress of the treatment, and
discontinuing radiation when the desired dose is achieved through the defined treatment region.

16. The method of claim 15, wherein the radiation is ionizing radiation.

17. The method of claim 16, wherein the ionizing radiation is x-ray radiation.

18. The method of claim 16, wherein the ionizing radiation is electron beam radiation.

19. The method of claim 16, wherein the ionizing radiation comprises alpha particles.

20. The method of claim 16, wherein the ionizing radiation comprises beta particles.

21. The method of claim 16, wherein the ionizing radiation comprises protons.

22. The method of claim 15, wherein the radiation is delivered using a miniature x-ray source and wherein the x-ray source is in the range 10 kV to 70 kV.

23. The method of claim 22, wherein the x-ray source is in the range of 10 kV to 30 kV.

24. The method of claim 15, wherein the step of defining the region to be treated comprises marking directly on a patient with a skin marking pen.

25. The method of claim 15, wherein the step of defining the region to be treated comprises imaging the region, displaying the region on a monitor connected to the computer, and marking electronically the image of the region on a monitor connected to the computer.

26. The method of claim 15, wherein the step of defining the region to be treated comprises imaging the region, displaying the region on a monitor connected to the computer, and marking electronically the image of the region on the monitor using a pointing device.

27. The method of claim 15, wherein the step of defining the region to be treated comprises automatic sensing of the condition of the skin surface via the computer, using visible characteristics of the condition.

28. The method of claim 15, wherein the step of defining the region to be treated comprises automatic sensing of the condition of the skin surface or near-skin via the computer, using visible characteristics of the condition, with physician modification using a monitor connected to the computer and displaying an image of the region, by means of a mouse, pen and tablet or pointing device connected to the computer.

29. The method of claim 15, wherein the treatment region is treated with the radiation by raster scan.

30. The method of claim 29, wherein the radiation is pulsed.

31. The method of 15, wherein the treatment region is treated with the radiation by a random pattern radiation beam delivery.

32. The method of claim 15, wherein the treatment region is treated with the radiation by patterned motion following a pre-defined pattern.

33. The method of claim 15, wherein the step of monitoring progress of the treatment comprises use of dosimeters in the radiation beam, to measure delivered radiation, with feedback from the dosimeters to the computer during the treatment and control of treatment parameters based on the feedback.

34. The method of claim 33, wherein the dosimeters are in an array across the region to be treated, closely adjacent to the region.

35. The method of claim 34, wherein the dosimeters are embedded in a flexible sheet having radiation attenuation density approximately the same as the dosimeters so that radiation is attenuated essentially uniformly across the beam on the treatment region.

36. The method of claim 33, wherein the dosimeters are positioned around the perimeter of the area to be treated, in the path of the radiation beam but essentially not shadowing the beam on the region to be treated.

37. The method of claim 33, wherein the dosimeters comprise semiconductor dosimeters.

38. The method of claim 15, wherein the step of monitoring the progress of the treatment comprises using range to tissue of the region and the output level of the radiation source, which is an electronic source, and integrating over time.

39. The method of claim 15, further including controlling depth of dose into the patient's skin so that some sub-regions of the region to be treated are subjected to deeper radiation treatment than other locations.

40. The method of claim 38, wherein the control of dose depth is by varying voltage to a controllable electronic miniature x-ray source supplying a beam of x-ray radiation.

41. The method of claim 15, further including displaying at least a portion of the treatment region on a monitor connected to the computer as the treatment progresses.

42. The method of claim 41, wherein the image on the monitor is obtained using a digital camera co-boresited with the radiation delivery source.

43. The method of claim 15, further including, during treatment, continuously re-acquiring the image of the region to be treated including during any movement of the region due to movement of the patient, using an image acquisition program run by the controller.

44. The method of claim 15, wherein radiation treatment is directed by a physician, including movement of the radiation to different positions on the treatment region, and including generating with the computer an automatic warning to the physician when a portion of the region is fully irradiated to dose prescription or when the radiation is off the treatment region.
